(19) United States
(12) Patent Application Publication
(10) Pub. No.: US 2026/0047847 A1
(43) Pub. Date: Feb. 19, 2026

(54) SYSTEMS, APPARATUSES, AND METHODS FOR CLIPS FORLEFT ATRIAL APPENDAGE CLOSURE

(71) Applicant: EDWARDS LIFESCIENCES CORPORATION, Irvine, CA (US)

(72) Inventors: Manouchehr A. Miraki, Laguna Hills, CA (US); Jason Thai Le, Garden Grove, CA (US); Da-Yu Chang, Irvine, CA (US); Cindy Woo, Irvine, CA (US); Maria L. Saravia, Irvine, CA (US); Amy E. Munnelly, Irvine, CA (US); Gidon Ofek, Newport Beach, CA (US); Irvin John Narciso, Tustin, CA (US)

(21) Appl. No.: 19/370,348

(22) Filed: Oct. 27, 2025

Related U.S. Application Data
(63) Continuation of application No. PCT/US2024/027395, filed on May 2, 2024.
(60) Provisional application No. 63/499,951, filed on May 3, 2023.

(51) Int. Cl. A61B 17/12

(52) U.S. Cl. CPC A61B 17/12122; A61B 17/12031; A61B 17/12131

(57) ABSTRACT

A clip may be configured to close the portion of the heart, to reduce blood flow therethrough as well as passage of clots or other undesired materials. The clip may be configured to close the left atrial appendage (LAA). The closure of the LAA may reduce the possibility of stroke or other maladies stemming from fluid flow with the LAA.

### CROSS REFERENCE TO RELATED APPLICATIONS

This application is a continuation of International Application No. PCT/US2024/027395, filed May 2, 2024, which claims the benefit of U.S. Patent Application No. 63/499,951, filed May 3, 2023, the entire disclosures all of which being incorporated by reference for all purposes.

### BACKGROUND

### Field

Various examples disclosed herein relate generally to clips for medical implementation. Some examples relate to clips for a left atrial appendage (LAA).

### Background

Cardiac arrhythmias are abnormal heart rhythms that can cause the heart to pump blood less effectively. Atrial fibrillation (AF) is one of the most common heart arrhythmia conditions. AF causes the left atrium to beat irregularly and reduces the efficiency of the “atrial kick” that helps to move blood into the left ventricle.

The left atrial appendage (LAA) is a muscular pouch located high on the free wall of the left atrium. The anatomy of the LAA is such that blood has a tendency to stagnate and form clots within the LAA. As blood flow is reduced with the progression of AF, the potential for clot formation increases tremendously.

Clots formed in the LAA can embolize into the bloodstream and move into the brain, where they can become lodged and eventually lead to stroke. It may be beneficial to close or occlude the LAA, to reduce the possibility of clots or other undesired materials from passing into the left atrium and into the bloodstream.

Left atrial appendage closure (also known as LAA closure or LAAC) is a minimally invasive procedure that is used to reduce the risk of stroke that comes as a result of atrial fibrillation.

### SUMMARY

Systems, apparatuses, and methods disclosed herein may be directed to clips for medical implementation, including clips for a portion of a heart. The clips may be configured to close the portion of the heart, to reduce blood flow therethrough as well as passage of clots or other undesired materials. In examples, the clips may be configured to close the left atrial appendage (LAA). The closure of the LAA may reduce the possibility of stroke or other maladies stemming from fluid flow with the LAA. In examples, the clips may be positioned exterior of the LAA, to extend over an outer surface of the LAA for closure.

In aspects, a clip for a portion of a heart is provided. The clip may comprise a first jaw extending from a first end portion to a second end portion along a length of the first jaw. The clip may comprise a second jaw extending from a first end portion to a second end portion along a length of the second jaw. The clip may comprise a spring configured to force the first jaw and the second jaw together to compress the portion of the heart with the first jaw and the second jaw. The clip may comprise one or more elongate couplers positioned on one or more of the first jaw or the second jaw and extending along the length of the respective first jaw or second jaw, the one or more elongate couplers configured to engage an expansion apparatus for the clip.

In aspects, a method. The method may comprise deploying a clip to close a portion of a heart. The clip may include a first jaw extending from a first end portion to a second end portion along a length of the first jaw. The clip may include a second jaw extending from a first end portion to a second end portion along a length of the second jaw. The clip may include a spring configured to force the first jaw and the second jaw together to compress the portion of the heart with the first jaw and the second jaw. The clip may include one or more elongate couplers positioned on one or more of the first jaw or the second jaw and extending along the length of the respective first jaw or second jaw, the one or more elongate couplers configured to engage an expansion apparatus for the clip.

In aspects, an expansion apparatus for a left atrial appendage clip is provided. The expansion apparatus may comprise an elongate shaft having a first end portion and a second end portion. The expansion apparatus may comprise an engagement portion positioned at the first end portion of the elongate shaft and having at least two arms, the at least two arms including a first arm for engaging a first jaw of the left atrial appendage clip and a second arm for engaging a second jaw of the left atrial appendage clip, the first arm configured to move relative to the second arm to open or close the left atrial appendage clip. The expansion apparatus may comprise a handle positioned at the second end portion of the elongate shaft. The expansion apparatus may comprise an actuator assembly configured to move the first arm relative to the second arm to open or close the left atrial appendage clip.

In aspects, a method. The method may comprise utilizing an expansion apparatus to deploy or capture a left atrial appendage clip. The expansion apparatus may include an elongate shaft having a first end portion and a second end portion. The expansion apparatus may include an engagement portion positioned at the first end portion of the elongate shaft and having at least two arms, the at least two arms including a first arm for engaging a first jaw of the left atrial appendage clip and a second arm for engaging a second jaw of the left atrial appendage clip, the first arm configured to move relative to the second arm to open or close the left atrial appendage clip. The expansion apparatus may include a handle positioned at the second end portion of the elongate shaft. The expansion apparatus may include an actuator assembly configured to move the first arm relative to the second arm to open or close the left atrial appendage clip.

### BRIEF DESCRIPTION OF THE DRAWINGS

FIG. 1 shows a cross sectional schematic view of a portion of a heart.

FIG. 2A illustrates a perspective view of a clip.

FIG. 2B illustrates a perspective view of the clip shown in FIG. 2A from an opposite side of the clip than shown in FIG. 2A.

FIG. 2C illustrates a side view of the clip shown in FIG. 2A.

FIG. 2D illustrates a top view of the clip shown in FIG. 2A.

FIG. 2E illustrates a proximal end view of the clip shown in FIG. 2A.

FIG. 2F illustrates a cross sectional view of the clip shown in FIG. 2A along line 2F-2F.

FIG. 2G illustrates a cross sectional view of the clip shown in FIG. 2A along line 2G-2G.

FIG. 2H illustrates an exploded view of the clip shown in FIG. 2A.

FIG. 3A illustrates a perspective view of an expansion apparatus.

FIG. 3B illustrates a close up perspective view of an engagement portion of an expansion apparatus.

FIG. 3C illustrates an exploded view of a portion of the expansion apparatus shown in FIG. 3A.

FIG. 3D illustrates an end view of the engagement portion of the expansion apparatus shown in FIG. 3A.

FIG. 4A illustrates a side view of the expansion apparatus shown in FIG. 3A engaged with a clip.

FIG. 4B illustrates a side view of the expansion apparatus shown in FIG. 3A engaged with a clip.

FIG. 4C illustrates a side view of a deployed clip.

FIG. 5 illustrates a top view of the clip shown in FIG. 2A.

FIG. 6 illustrates a perspective view of a clip.

FIG. 7A illustrates a perspective view of a clip.

FIG. 7B illustrates a perspective view of the clip shown in FIG. 7A from an opposite side than shown in FIG. 7A.

FIG. 8A illustrates a perspective view of a clip.

FIG. 8B illustrates an end view of the clip shown in FIG. 8A.

FIG. 9A illustrates a proximal end perspective view of a clip.

FIG. 9B illustrates a distal end perspective view of the clip shown in FIG. 9A.

FIG. 9C illustrates a top perspective view of the clip shown in FIG. 9A.

FIG. 10A illustrates a perspective view of a clip.

FIG. 10B illustrates a proximal end view of the clip shown in FIG. 10A.

FIG. 10C illustrates a cross sectional view of the clip shown in FIG. 10A along line 10C-10C.

FIG. 11A illustrates a perspective view of a clip.

FIG. 11B illustrates a perspective view of the clip shown in FIG. 11A from an opposite side than shown in FIG. 11A.

FIG. 11C illustrates a proximal end view of the clip shown in FIG. 11A.

FIG. 12A illustrates a perspective view of an expansion apparatus.

FIG. 12B illustrates a perspective view of an engagement portion of the expansion apparatus shown in FIG. 12A.

FIG. 12C illustrates an exploded view of a portion of the engagement apparatus shown in FIG. 12A.

FIG. 12D illustrates a perspective view of the expansion apparatus shown in FIG. 12A engaged with a clip.

FIG. 13A illustrates a perspective view of an engagement portion of an expansion apparatus.

FIG. 13B illustrates a perspective view of the engagement portion of the expansion apparatus shown in FIG. 13A from an opposite side than shown in FIG. 13A.

FIG. 13C illustrates a perspective view of the engagement portion shown in FIG. 13A engaged with a clip.

FIG. 13D illustrates a perspective view of the engagement portion shown in FIG. 13A engaged with a clip from an opposite side than shown in FIG. 13A.

FIG. 14A illustrates a perspective view of a clip.

FIG. 14B illustrates a cross sectional view of the clip shown in FIG. 14A along line 14B-14B.

FIG. 15A illustrates a perspective view of a clip.

FIG. 15B illustrates a cross sectional view of the clip shown in FIG. 15A along line 15B-15B.

FIG. 16A illustrates a perspective view of a clip.

FIG. 16B illustrates a cross sectional view of the clip shown in FIG. 16A along line 16B-16B.

### DETAILED DESCRIPTION

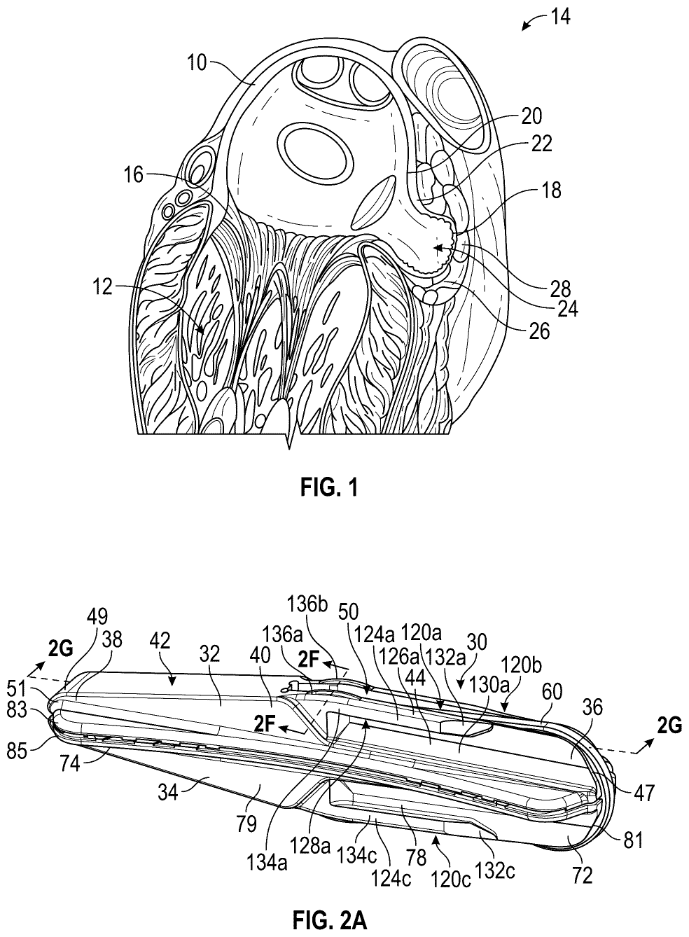

FIG. 1 illustrates a cross sectional view of a left atrium 10 and a left ventricle 12 of an individual's heart 14. The left atrium 10 is configured to fill with blood to pass into the left ventricle 12 via the mitral valve 16 during the cardiac cycle. The left atrial appendage (LAA) 18 protrudes from the outer wall 20 of the left atrium 10 and includes an ostium 22 and a cavity 24 extending from the ostium 22. A LAA wall 26 may surround the cavity 24 and may form an outer surface 28 of the LAA 18. The LAA 18 has a pouch shape extending from the left atrium 10. The cavity 24 may be configured to fill with blood, allowing the LAA to serve as a decompression chamber during systole and when pressure otherwise increases in the left atrium 10.

In certain individuals, blood may stagnate and form clots within the LAA 18. Clots or other undesired materials stemming from the LAA 18 may travel into the bloodstream, producing a variety of maladies including strokes. It may thus be beneficial to close the LAA 18, to reduce the possibility of clots or other undesired material from producing such maladies.

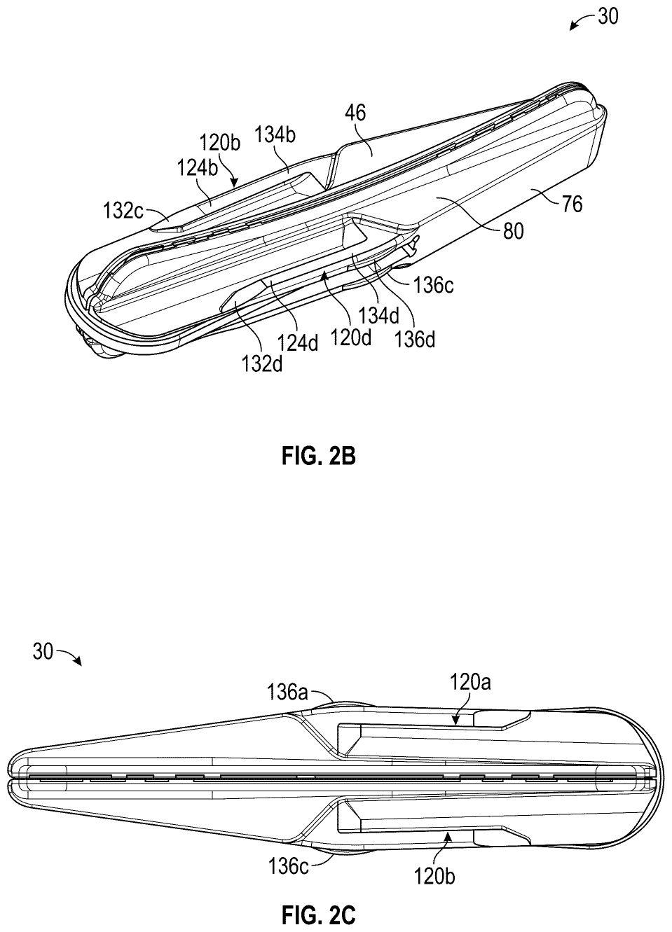

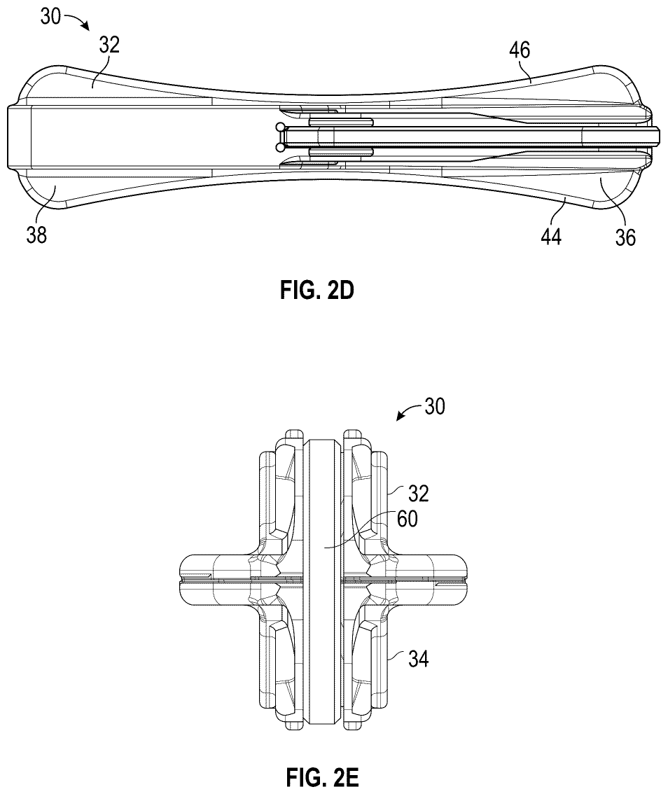

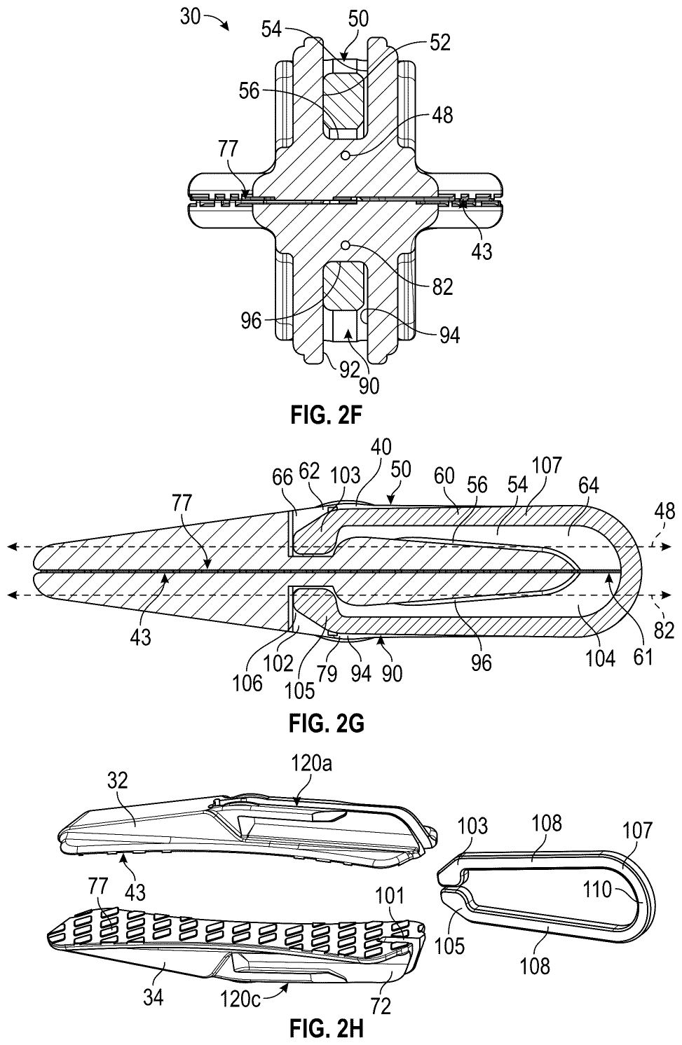

FIGS. 2A-2H illustrate an example of a clip 30 that may be utilized for a portion of a heart. The clip 30 may be configured to close a LAA 18, to reduce the possibility of clots or other undesired materials stemming from the LAA 18 from traveling into the bloodstream.

The clip 30 may include a first jaw 32 and a second jaw 34. The first jaw 32 may extend from a first end portion 36 to a second end portion 38 along a length of the first jaw 32. The first jaw 32 may have an elongate shape. The first jaw 32 may be configured as an elongate beam. The first jaw 32 may extend along a longitudinal axis 48 (marked in FIGS. 2F and 2G). The first jaw 32 may have a central portion 40 between the first end portion 36 and the second end portion 38.

The first end portion 36 may comprise a proximal end portion of the clip 30. The second end portion 38 may comprise a distal end portion of the clip 30.

The first jaw 32 may include an outer surface 42, a compression surface 43 (marked in FIGS. 2F and 2G), and two side surfaces 44, 46 (with side surface 46 shown in FIG. 2B) each extending from the compression surface 43 to the outer surface 42. The two side surfaces 44, 46 may face opposite each other. The compression surface 43 may face opposite the outer surface 42 and towards the second jaw 34.

The first jaw 32 may include a first end surface 47 positioned at the first end portion 36 of the first jaw 32. The first jaw 32 may include a second end surface 49 positioned at the second end portion 38 of the first jaw 32. The first end surface 47 may comprise a proximal end surface of the first jaw 32 and the second end surface 49 may comprise a distal end surface of the first jaw 32.

In examples, the second end portion 38 of the first jaw 32 may be tapered, such that a rounded tip 51 of the first jaw 32 is provided. The rounded tip 51 of the first jaw 32 may allow the first jaw 32 to be atraumatic to a patient's body upon insertion into the body and deployment to a desired location. In examples, the first end portion 36 of the first jaw 32 may be tapered.

In examples, one or more of the side surfaces 44, 46 may be curved. The curvature may have a variety of forms and may comprise a concave curvature relative to the first jaw 32 (as shown in FIG. 2D for example). Referring to FIG. 2D, for example, the side surface 44 being curved concave relative to the first jaw 32 is shown. The curvature may have a constant radius of curvature, or may have a varied radius of curvature in examples. The curvature may extend from the first end portion 36 of the clip 30 to the second end portion 38 of the clip 30 or may extend for a portion of the clip 30. In examples, the side surface 46 may be curved concave relative to the first jaw 32. The curvature may comprise a same curvature as the side surface 44 or may comprise a different curvature.

A curvature of one or more of the side surfaces 44, 46 may allow the clip 30 to contour to a shape of a portion of the heart upon deployment. For example, the surface 44 may contour to a shape of the wall of the left atrium upon deployment. The surface 46 may contour to the shape of the wall of the left atrium upon the clip 30 being deployed in an opposite orientation (in which the side surface 46 faces the wall of the left atrium). Other configurations of curvature may be utilized in examples.

The outer surface 42 of the first jaw 32 may include a channel 50. FIG. 2F illustrates a cross sectional view of the clip 30 along line 2F-2F in FIG. 2A, and FIG. 2G illustrates a cross sectional view of the clip 30 along line 2G-2G in FIG. 2A. Referring to FIGS. 2F and 2G, the channel 50 may include a first side wall 52, a second side wall 54, and a lower wall 56 of the channel 50. The upper portion of the channel 50 may remain open for a spring 60 to pass through. The channel 50 may extend from the central portion 40 of the first jaw 32 to the first end portion 36 of the first jaw 32. In examples, the channel 50 may include a cut out portion 61 of the compression surface 43 at the first end portion 36 to allow for the spring 60 to pass through the compression surface 43.

The channel 50 may include a first end 62 and a second end 64, with the second end 64 being opened to allow for the spring 60 to pass through.

A coupler 66 may be positioned at the first end 62 of the channel 50 and configured to receive an end of the spring 60. The coupler 66 may comprise a cavity in the first jaw 32 or may have another configuration as desired.

The second jaw 34 may extend from a first end portion 72 to a second end portion 74 along a length of the second jaw 34. The second jaw 34 may have an elongate shape. The second jaw 34 may be configured as an elongate beam. The second jaw 34 may extend along a longitudinal axis 82 (marked in FIGS. 2F and 2G). The second jaw 34 may have a central portion 79 between the first end portion 72 and the second end portion 74.

The first end portion 72 may comprise a proximal end portion of the clip 30. The second end portion 74 may comprise a distal end portion of the clip 30.

The second jaw 34 may include an outer surface 76 (marked in FIG. 2B), a compression surface 77 (marked in FIG. 2H), and two side surfaces 78, 80 (with side surface 80 shown in FIG. 2B) each extending from the compression surface 77 to the outer surface 76. The two side surfaces 78, 80 may face opposite each other. The compression surface 77 may face opposite the outer surface 76 and towards the first jaw 32.

The second jaw 34 may include a first end surface 81 positioned at the first end portion 72 of the second jaw 34. The second jaw 34 may include a second end surface 83 positioned at the second end portion 74 of the second jaw 34. The first end surface 81 may comprise a proximal end surface of the second jaw 34 and the second end surface 83 may comprise a distal end surface of the second jaw 34.

In examples, the second end portion 74 of the second jaw 34 may be tapered, such that a rounded tip 85 of the second jaw 34 is provided. The rounded tip 85 of the second jaw 34 may allow the second jaw 34 to be atraumatic to a patient's body upon insertion into the body and deployment to a desired location. In examples, the first end portion 72 of the second jaw 34 may be tapered.

In examples, one or more of the side surfaces 78, 80 may be curved. The one or more side surfaces 78, 80 may be curved in a similar manner as described regarding the curvature of the side surfaces 44, 46. The curvature of one or more of the side surfaces 78, 80 may be different from the curvature of the side surfaces 44, 46 in examples.

The outer surface 76 of the second jaw 34 may include a channel 90. Referring to FIGS. 2F and 2G, the channel 90 may include a first side wall 92, a second side wall 94, and a lower wall 96 of the channel 90. The upper portion of the channel 90 may remain open for the spring 60 to pass through. The channel 90 may extend from the central portion 79 of the second jaw 34 to the first end portion 72 of the second jaw 34. In examples, the channel 90 may include a cut out portion 101 (marked in FIG. 2H) of the compression surface 77 at the first end portion 72 to allow for the spring 60 to pass through the compression surface 77.

The channel 90 may include a first end 102 and a second end 104, with the second end 104 being opened to allow for the spring 60 to pass through.

A coupler 106 may be positioned at the first end 102 of the channel 90 and configured to receive an end of the spring 60. The coupler 106 may comprise a cavity in the second jaw 34 or may have another configuration as desired.

In examples, the compression surfaces 43, 77 of the respective jaws 32, 34 may be coated with a medical grade soft material to make the compression surfaces of the jaws 32, 34 atraumatic if desired.

Referring to FIG. 2A, the spring 60 may be configured to force the first jaw 32 and the second jaw 34 together to compress a portion of the heart with the first jaw 32 and the second jaw 34. The spring 60 may extend over the outer surface 42 of the first jaw 32 and the outer surface 76 of the second jaw 34 and may be configured to force the first jaw 32 and the second jaw 34 together to compress the portion of the heart between the compression surface 43 of the first jaw 32 (marked in FIG. 2G) and the compression surface 77 of the second jaw 34 (marked in FIG. 2H). The spring 60, for example may have a “C” shape with a first end 103 and a second end 105 and a loop 107 coupled to the first end 103 and the second end 105 (marked in FIG. 2H). The loop 107 may include straight portions 108 coupled to a curved portion 110 forming the curve of the “C” shape. The first end 103 of the spring 60 may be coupled to the central portion 40 of the first jaw 32 (marked in FIG. 2G) and the second end 105 of the spring 60 may be coupled to the central portion 79 of the second jaw 34 (marked in FIG. 2G), and the loop 107 may extend towards the first end portion 36 of the first jaw 32 and the first end portion 72 of the second jaw 34.

The loop 107 may extend within the channel 50 of the first jaw 32 and the channel 90 of the second jaw 34. The loop 107 may be positioned within the channel 50 of the first jaw 32 and the channel 90 of the second jaw 34 between the respective side walls 52, 54 of the channel 50 and the side walls 92, 94 of the channel 90 (marked in FIG. 2F). The loop 107 may be spaced from the respective lower walls 56, 96 of the channels 50, 90 such that the loop 107 may move towards the respective lower walls 56, 96 upon the clip 30 being in an opened state. The spring 60, for example, may pivot with respect to the lower walls 56, 96 upon the clip 30 being in an opened state.

The position of the loop 107 within the channels 50, 90 may allow for expansion of the clip 30. The position of the loop 107 within the channels 50, 90 may reduce the possibility of twisting of the spring 60 during opening or closing of the clip 30.

The loop 107 may reside within the channels 50, 90 continuously and may reduce the possibility of the jaws 32, 34 from moving distally and side to side.

The spring 60 may extend within the plane of movement of the first jaw 32 and the second jaw 34.

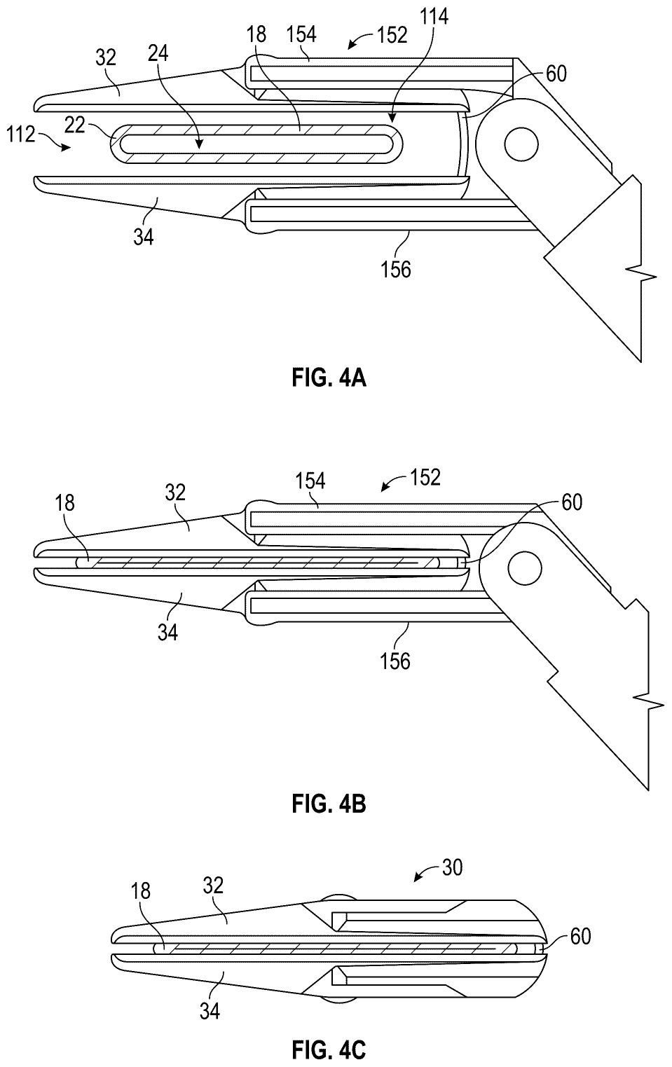

The spring 60 may be configured to allow the clip 30 to move from an opened state to a closed state, yet force the clip 30 towards the closed state. The spring 60 accordingly may provide a force that moves the compression surfaces 43, 77 of the first jaw 32 and second jaw 34 towards each other to compress a portion of the heart therein. The spring 60 may be configured to keep the jaws 32, 34 under positive compression at all times, even at rest. The spring 60 may have a “C” shape to allow the second end portions 38, 74 of the first jaw 32 and the second jaw 34 respectively to form an axial opening 112 (marked in FIG. 4A) for a space 114 (marked in FIG. 4A) between the first jaw 32 and the second jaw 34 for receiving the portion of the heart. The space 114 may comprise a compression channel between the first jaw 32 and the second jaw 34. The axial opening 112 may comprise an opening at the second end portions 38, 74 of the first jaw 32 and second jaw 34. The clip 30 may be positioned in an opened state and with the portion of the heart slid through the axial opening 112 and into the space 114. In examples, other methods of entry into the space 114 may be provided (e.g., along an axis of the LAA).

The second end portion 38 of the first jaw 32 and the second end portion 74 of the second jaw 34 may form an open end of the clip 30. The first end portion 36 of the first jaw 32 and the first end portion 72 of the second jaw 34 may form a closed end of the clip 30.

The loop 107 of the spring 60 may form a boundary of the space 114 between the first jaw 32 and the second jaw 34. The compression channel may be closed at the first end portions 36, 72 of the first jaw 32 and second jaw 34. The loop 107 of the spring 60 may close the space 114 at the first end portion 36 of the first jaw 32 and the first end portion 72 of the second jaw 34. The loop 107 accordingly may prevent the clip 30 from sliding distally with respect to the LAA 18 upon deployment, and may prevent the tissue of the LAA 18 from protruding further than the loop 107 upon compression of the LAA 18.

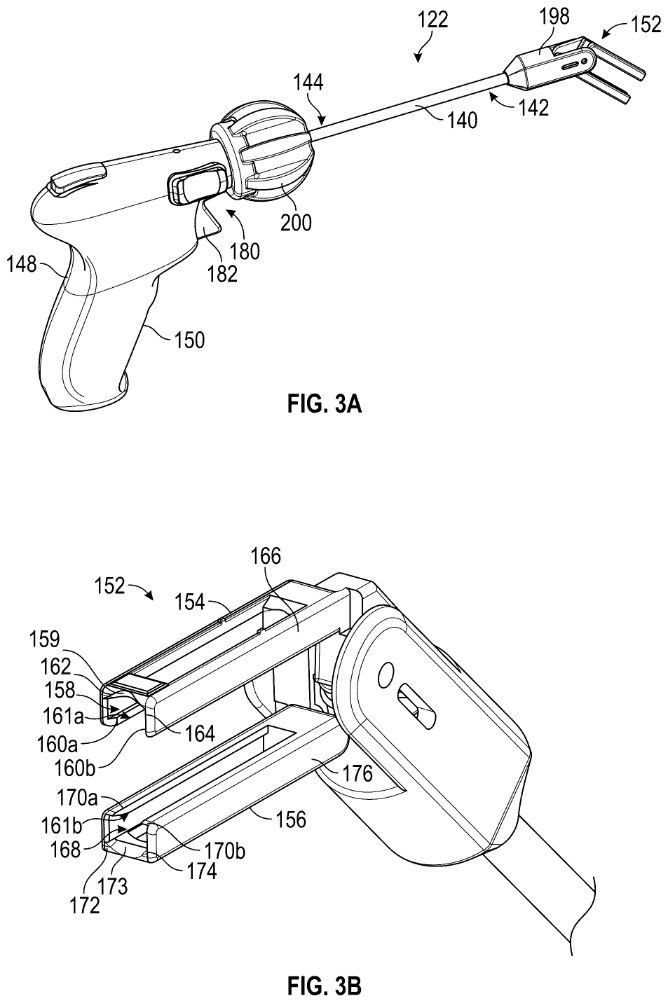

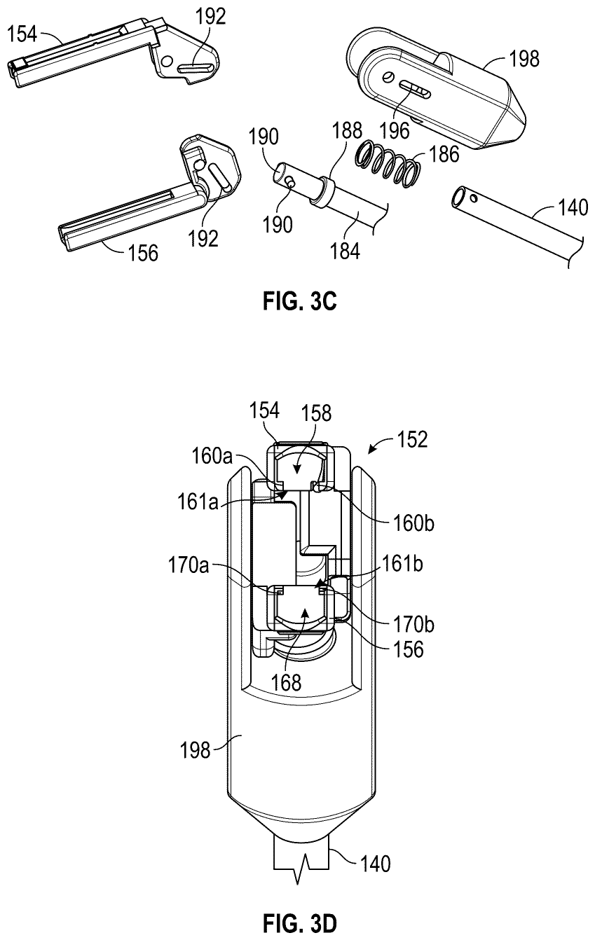

In examples, the clip 30 may include one or more elongate couplers 120a-d (marked in FIGS. 2A and 2B). The elongate couplers 120a-d may be positioned on one or more of the first jaw 32 or the second jaw 34 and may extend along a length of the respective first jaw 32 or second jaw 34. The elongate couplers 120a-d may extend along the longitudinal dimension of the respective first jaw 32 or second jaw 34. In examples, the elongate couplers 120a-d may extend parallel with a respective one of the longitudinal axis 48 of the first jaw 32 or the longitudinal axis 82 of the second jaw 34 (marked in FIG. 2G). The one or more elongate couplers 120a-d may be configured to engage an expansion apparatus 122 (marked in FIG. 3A) for the clip 30.

In examples, the one or more elongate couplers 120a-d may include one or more protrusions 124a-d (marked in FIGS. 2A and 2B). The protrusion 124a may extend from the side surface 44 of the first jaw 32 and the protrusion 124b may extend from the side surface 46 of the first jaw 32 (marked in FIG. 2B). The protrusion 124c may extend from the side surface 78 of the second jaw 34 and the protrusion 124d may extend from the side surface 80 of the second jaw 34 (marked in FIG. 2B).

The protrusions 124a-d may extend along the length of the respective first jaw 32 or second jaw 34. The protrusions 124a-d may form rails extending along the length of the respective first jaw 32 or second jaw 34. An outer surface 126a of the protrusion 124a may comprise a portion of the outer surface 42 of the first jaw 32. An inner surface 128a of the protrusion 124a may face opposite the outer surface 126a and may face towards a channel 130a extending along the side surface 44 of the first jaw 32. The channel 130a may extend along the length of the first jaw 32. The protrusion 124b may be similarly configured on the side surface 46 of the first jaw 32. The protrusions 124c, d may be similarly configured on the respective side surfaces 78, 80 of the second jaw 34. In examples, the protrusions 124a-d may have similar configurations as each other or differing configurations.

The protrusions 124a-d may each extend proximally from the central portions 40, 79 of the respective first jaw 32 and second jaw 34 towards the first end portions 36, 72 of the respective first jaw 32 and second jaw 34. As shown in FIGS. 2A and 2B, the protrusions 124a-d may extend to a mid point between the central portions 40, 79 and the respective first end surfaces 47, 81. The protrusions 124a-d may each include a respective side surface 132a-d that may be angled or ramped at the mid point between the central portions 40, 79 and the respective first end surfaces 47, 81. The angled side surface 132a-d may allow for improved sliding engagement with the expansion apparatus 122.

In examples, the protrusions 124a-d may include a respective elongate side surface 134a-d (marked in FIGS. 2A and 2B) that may comprise a portion of the respective side surface 44, 46, 78, 80.

The configuration of the one or more elongate couplers 120a-d may vary in examples.

In examples, the clip 30 may include one or more securement bodies 136a-d. The securement bodies 136a-d may be utilized for securement with an expansion apparatus 122 in examples. The securement bodies 136a-d may comprise protrusions that may be engaged by the expansion apparatus or may have other configurations in examples. The protrusions may comprise bumps as shown in FIGS. 2A and 2B for example. The bumps may extend from the respective outer surfaces 42, 76 of the first jaw 32 and second jaw 34.

The configuration of the securement bodies 136a-d may vary in examples.

FIG. 2C illustrates a side view of the clip 30. FIG. 2D illustrates a top view of the clip 30. FIG. 2E illustrates a proximal end view of the clip 30.

FIG. 2H illustrates an exploded view of the clip 30. In examples, one or more of the compression surface 43 of the first jaw 32 or the compression surface 77 of the second jaw 34 may include a textured surface that may enhance grip with the portion of the heart being occluded. In examples, one or more of the compression surfaces 43, 77 may comprise smooth surfaces.

In examples, the one or more elongate couplers 120a-d may be configured to slidably engage with an expansion apparatus 122. The one or more elongate couplers 120a-d may be configured to engage the expansion apparatus 122 advanced in a direction from the proximal end or closed end of the clip 30 towards the distal end or open end of the clip. For example, the one or more elongate couplers 120a-d may be configured such that the expansion apparatus 122 is advanced from the first end portions 36, 72 of the jaws 32, 34 towards the second end portions 38, 74 of the jaws 32, 34.

Features of the clip 30 may be utilized solely or in combination with any other example herein.

FIG. 3A illustrates a perspective view of an expansion apparatus 122 that may be utilized in examples. The expansion apparatus 122 may include an elongate shaft 140 having a first end portion 142 or distal end portion and a second end portion 144 or proximal end portion. The elongate shaft 140 may comprise a rigid shaft or may comprise a flexible shaft in examples. The elongate shaft 140 may be configured for one or more components of an actuator assembly to pass therethrough, to extend to the engagement portion 152 in examples.

The expansion apparatus 122 may include a handle 148 in examples. The handle 148 may be positioned at the second end portion 144 or proximal end portion of the elongate shaft 140. The handle 148 may include a grip portion 150 for a user to grip in examples.

The expansion apparatus 122 may include an engagement portion 152. The engagement portion 152 may be configured to engage the clip 30 in examples. The engagement portion 152 may be positioned at the first end portion 142 of the elongate shaft 140 in examples.

FIG. 3B illustrates a detail perspective view of the engagement portion 152. The engagement portion 152 may have a first arm 154 and a second arm 156. The first arm 154 may be configured to engage the first jaw 32 of the clip 30, and the second arm 156 may be configured to engage the second jaw 34 of the clip 30. The first arm 154 may be configured to move relative to the second arm 156 to open or close the clip 30.

The first arm 154 may include a channel 158 for engaging the first jaw 32 of the clip 30. The channel 158 may extend along a length of the first arm 154. One or more rails 160a, b may extend along the channel 158 and may be configured to retain the first jaw 32 within the channel 158. The rails 160a, b may comprise opposed rails on opposite sides of the channel 158. The rails 160a, b may bound an inner opening 161a of the first arm 154 that faces towards the second arm 156. The first arm 154 may include a distal tip 162 that may include an opening 164 for the channel 158.

The first arm 154 may extend from a proximal portion 166 of the first arm 154 to the distal tip 162 of the first arm 154.

In examples, an upper surface of the first arm 154 may include a recess 159. The recess 159 may be configured to receive one or more securement bodies 136a, b of the first jaw 32 in examples.

The second arm 156 may be configured similarly as the first arm 154. For example, the second arm 156 may include a channel 168 for engaging the second jaw 34 of the clip 30. The channel 168 may extend along a length of the second jaw 34. One or more rails 170a, b may extend along the channel 168 and may be configured to retain the second jaw 34 within the channel 168. The rails 170a, b may comprise opposed rails on opposite sides of the channel 168. The rails 170a, b may bound an inner opening 161b of the second arm 156 that faces towards the first arm 154. The second arm 156 may include a distal tip 172 that may include an opening 174 for the channel 168.

The second arm 156 may extend from a proximal portion 176 of the second arm 156 to the distal tip 172 of the second arm 156.

In examples, a lower surface of the second arm 156 may include a recess 173. The recess 173 may be configured to receive one or more securement bodies 136c, d of the second jaw 34 in examples.

The first arm 154 and the second arm 156 may extend parallel with each other. The first arm 154 and second arm 156 may be configured to be positioned in a plane of movement of the first arm 154 and the second arm 156, with elongate inner openings 161a, b of the channels 158, 168 facing towards each other.

The first arm 154 may be configured for the first jaw 32 to be slid proximally in a direction from the distal tip 162 towards the proximal portion 166 of the first arm 154. The second arm 156 may be configured for the second jaw 34 to be slid proximally in a direction from the distal tip 172 of the second arm 156 towards the proximal portion 176 of the second arm 156. The rails 160a, b of the first arm 154 may engage the elongate couplers 120a, b of the first jaw 32. The rails 170a, b of the second arm 156 may engage the elongate couplers 120c, d of the second jaw 34. The rails 160a, b, for example, may overlap the inner surfaces of the elongate couplers 120a, b of the first jaw 32 and the rails 170a, b may overlap the inner surfaces of the elongate couplers 120c, d. The elongate couplers 120a-d may be inserted through the openings 164, 174 of the respective arms 154, 156.

The elongate couplers 120a-d may be slid along the channels 158, 168 in a proximal direction until the respective securement bodies 136a-d engage with the recesses 159, 173. Such a configuration is represented in FIG. 4A for example.

The first arm 154 may be configured to be moved away from the second arm 156 to open the clip 30. The first arm 154 may be configured to be moved towards the second arm 156 to close the clip 30.

In examples, an actuator assembly may be configured to move the first arm 154 relative to the second arm 156 to open or close the clip 30. The actuator assembly may have a variety of forms in examples. For example, referring to FIG. 3A, an actuator assembly 180 may include a control device 182. The control device 182 may comprise a device for a user to operate to move the first arm 154 relative to the second arm 156. The control device 182 may comprise a button or trigger or may have another form in examples (knobs, sliders, electronic switches, among others). The control device 182 may control an actuator body 184 (marked in FIG. 3C).

FIG. 3C illustrates an exploded view of the engagement portion 152 of the expansion apparatus 122 and components of the actuator assembly 180. A distal end portion of the actuator body 184 is shown. The actuator body 184 may extend within the interior of the elongate shaft 140 for axial control and movement of the actuator body 184 relative to the elongate shaft 140. A proximal end portion of the actuator body 184 may extend to and may be controlled by the control device 182. In examples, the actuator assembly 180 may include a spring 186 that may bias the actuator body 184 in a distal direction. The spring 186, for example, may press against a spring retainer 188 that may be positioned upon the actuator body 184.

The actuator body 184 may include couplers 190 that may be configured to engage with engagement surfaces 192 of the first arm 154 and the second arm 156. The couplers 190 may comprise pins or may have another configuration as desired. The engagement surfaces 192 may comprise slots or may have another configuration for engagement with the couplers 190 (e.g., the engagement surfaces 192 may comprise pins and the couplers 190 may comprise slots as desired). The couplers 190 may pass through channels formed by the engagement surfaces 192 to be engaged by slots 196 in an adaptor body 198. The engagement surfaces 192 and slots 196 may be oriented relative to each other such that proximal movement of the actuator body 184 may cause the arms 154, 156 to move away from each other, and distal movement of the actuator body 184 may cause the arms 154, 156 to move towards each other. For example, the control device 182 shown in FIG. 3A may be retracted to cause the actuator body 184 to move proximally. The proximal movement of the actuator body 184 may cause the arms 154, 156 to move to an opened position and open the clip 30. The spring 186 may bias the actuator body 184 distally to move the arms 154, 156 to the closed position and close the clip 30 upon the control device 182 being released.

In examples, the arms 154, 156 may extend parallel with each other in the closed state. The actuator assembly 180 may be configured to cause the arms 154, 156 to extend parallel with each other in the opened state. In examples, the actuator assembly 180 may be configured to cause the arms 154, 156 to angle with respect to each other in an opened state.

The adaptor body 198 may be configured to pivot about the elongate shaft 140 or with the elongate shaft 140 in examples. A pivot coupling of the adaptor body 198 to the elongate shaft 140 may be utilized. The pivotal movement of the adaptor body 198 may cause the engagement portion 152 to pivot about the elongate shaft 140. In examples, a control device 200 (as marked in FIG. 3A) may be utilized to pivot the adaptor body 198 relative to the elongate shaft 140 or with the elongate shaft 140. The control device 200 may comprise a knob, yet other configurations may be utilized as desired.

FIG. 3D illustrates an end view of the engagement portion 152.

FIGS. 4A-4C illustrate an exemplary deployment of the clip 30 utilizing the expansion apparatus 122. The clip 30 may be engaged by the engagement portion 152 of the expansion apparatus 122. The elongate couplers 120a-d, for example, may be positioned within the channels 158, 168 shown in FIG. 3B for example. The clip 30 may be held in an opened state.

The expansion apparatus 122 may be advanced to a desired deployment location. Access may be provided to the LAA 18 in a variety of manners, including a transcatheter access or a surgical access such as a thoracotomy, mini thoracotomy, or other methods of access.

The clip 30 may be advanced in an opened state over the portion of the heart to be compressed, for example, over the ostium 22 of the left atrial appendage (LAA) 18. The LAA 18 may be slid through the axial opening 112 although other methods of positioning may be utilized (e.g., the clip 30 may be slid along the axis of the LAA 18 in examples).

Referring to FIG. 4B, the engagement portion 152 may be moved to the closed position to compress the LAA 18. The engagement portion 152 may be reopened as desired to reposition the clip 30 and for the clip 30 to be subsequently closed in the desired position. With the clip 30 in the desired position, the engagement portion 152 may be retracted in a proximal direction. The sliding engagement of the elongate couplers 120a-d with the arms 154, 156 may allow the arms 154, 156 to be slid off of the couplers 120a-d proximally with the clip 30 remaining in position.

FIG. 4C illustrates a resulting position of the clip 30 occluding the LAA 18.

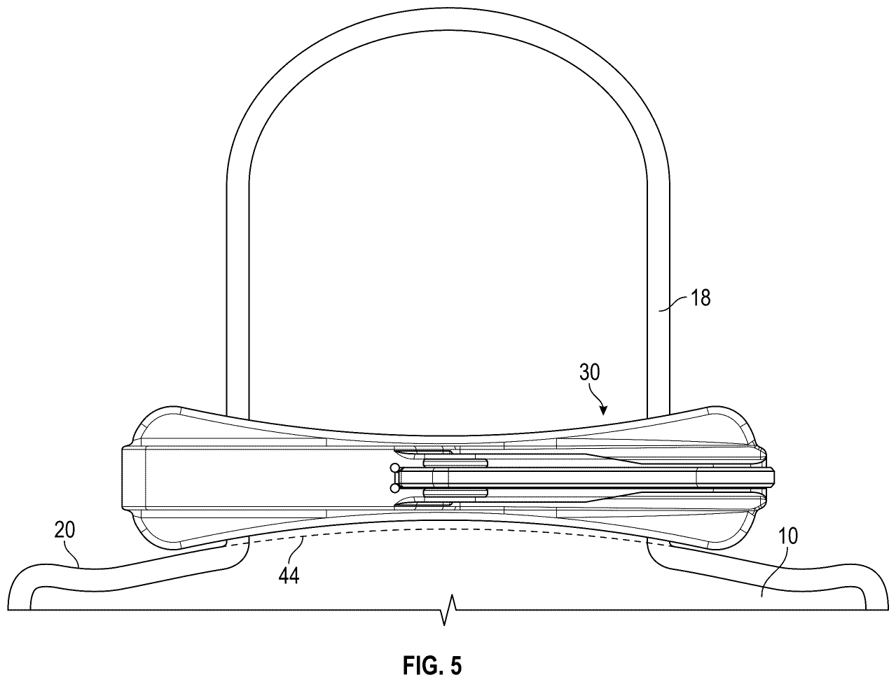

FIG. 5 illustrates a top view of the clip 30 occluding the LAA 18. The side surface 44 of the clip 30 may contour to a shape of the left atrium 10 in examples.

If desired, the clip 30 may be captured utilizing the expansion apparatus 122. The expansion apparatus 122, for example, may be slid onto the elongate couplers 120a-d in vivo. The arms 154, 156 may be opened to move the clip 30 to an opened state for release from the LAA 18. The clip 30 may be repositioned or entirely removed from the LAA 18 as desired. The expansion apparatus 122 may be utilized for deployment or capture of the clip 30.

Features of the expansion apparatus 122 may be utilized solely or in combination with any other example herein.

The clip 30 may be utilized to close the LAA 18, yet in examples other portions of a heart may be clipped or closed via use of the clip 30. In examples, other portions of a body, such as a tubular vessel or other portions of a body may be closed with the clip 30. Deployment may be via an expansion apparatus or via another method as desired.

Variations in the configuration of the clip 30 may be provided as desired.

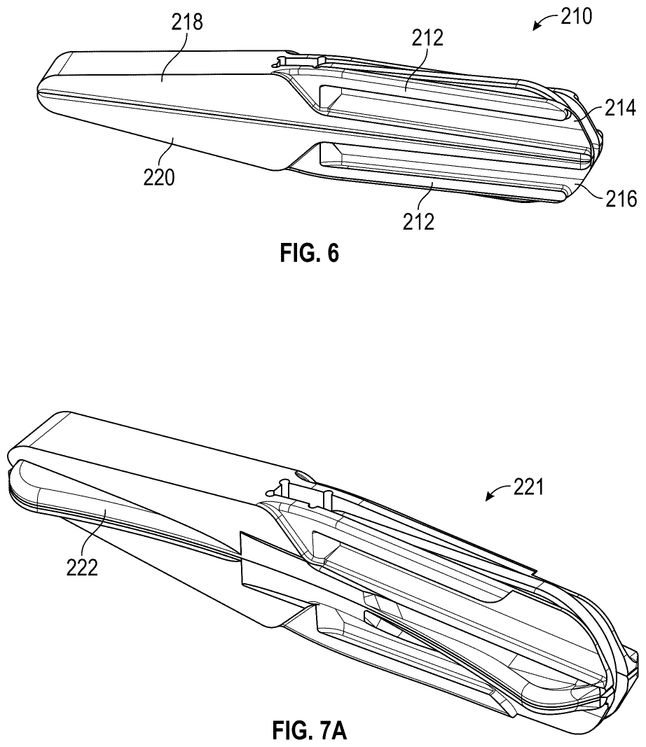

FIG. 6, for example, illustrates an example of a clip 210 in which the elongate couplers 212 extend to the proximal end surfaces 214, 216 of the respective first jaw 218 and second jaw 220. The greater length of the elongate couplers 212 in the example shown in FIG. 6 relative to the example shown in FIG. 1 may increase the length of the elongate couplers 212 that is engaged by the expansion apparatus 122. Features described in regard to the clip 30 may otherwise be utilized with the clip 210.

The features of the clip 210 may be utilized solely or in combination with any other example disclosed herein.

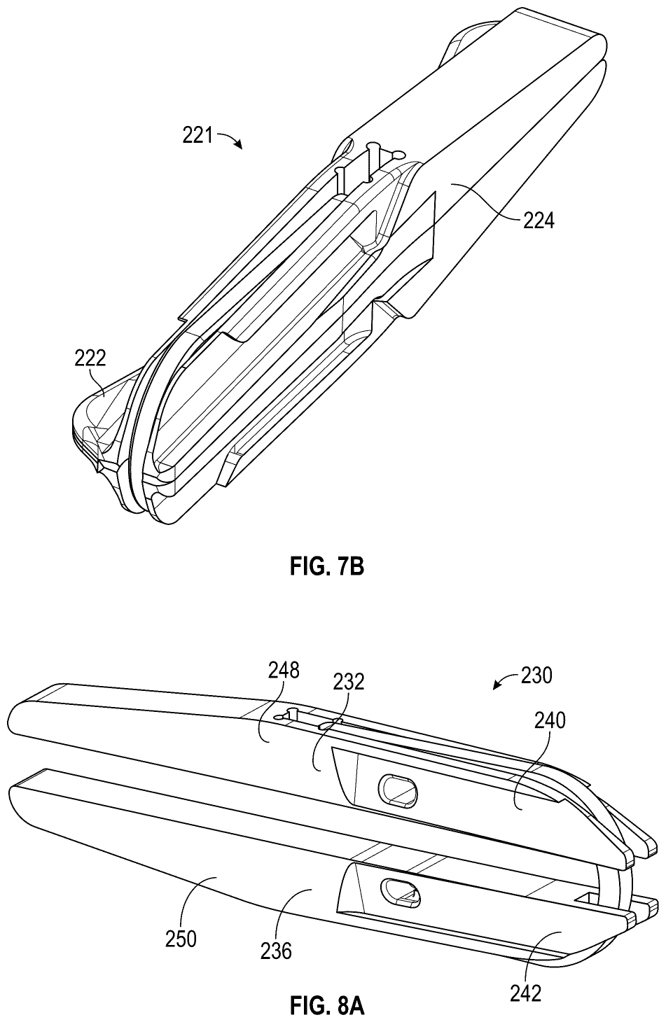

FIGS. 7A and 7B illustrate an example of a clip 221 having a first side surface 222 with a curvature, and an opposite second side surface 224 (marked in FIG. 7B) that lacks a curvature. The second side surface 224 may be flat or linear in examples. The first side surface 222 may be deployed to face the left atrium upon deployment. In examples, the second side surface 224 may be configured to face the left atrium upon deployment. Such a configuration of clip 221 may provide a user with a selection of whether to face a curved side surface 222 towards the left atrium or face a flat side surface 224 towards the left atrium in examples. Features described in regard to the clip 30 may otherwise be utilized with the clip 221.

The features of the clip 221 may be utilized solely or in combination with any other example disclosed herein.

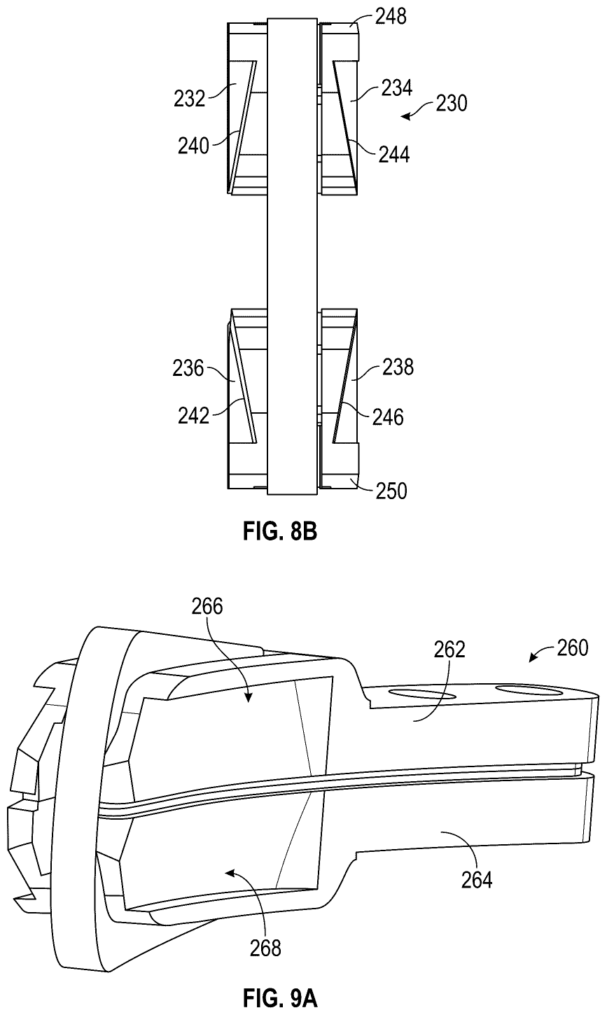

FIGS. 8A and 8B illustrate an example of a clip 230 having side surfaces 232, 234, 236, 238 with respective channels 240, 242, 244, 246 that are angled relative to a plane of closure of the clip 230. The channels 240, 242, 244, 246 may comprise elongate wedges extending along the lengths of the respective jaws 248, 250. The shape of the channels 240, 242, 244, 246 may serve to orient the clip 230 with respect to the expansion apparatus 122. For example, the shape of the channels 240, 242, 244, 246 may align the clip 230 with the arms 154, 156 of the engagement portion 152. Features described in regard to the clip 30 may otherwise be utilized with the clip 230.

The features of the clip 230 may be utilized solely or in combination with any other example disclosed herein.

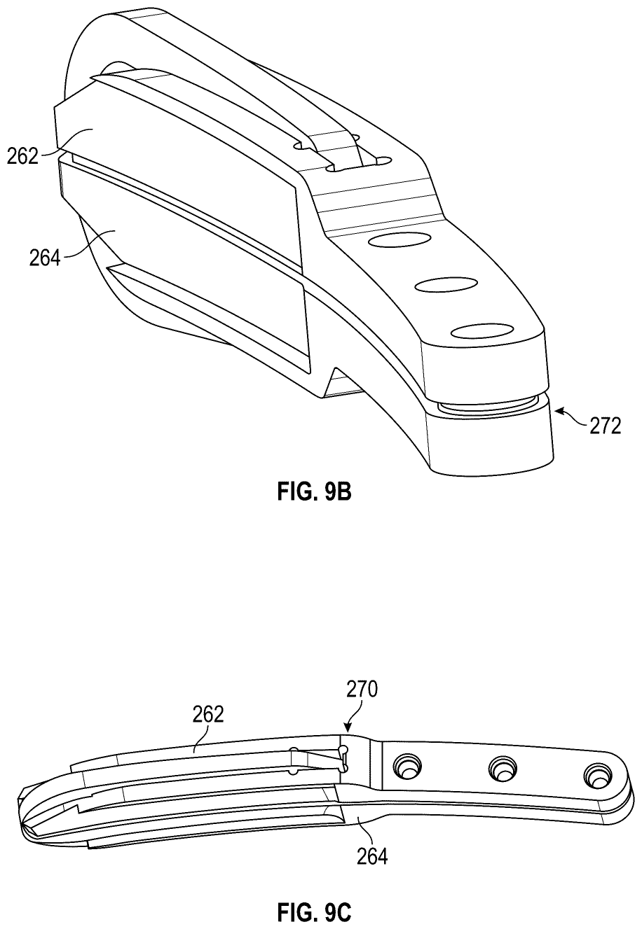

FIGS. 9A-9C illustrate a variation of the clip shown in FIGS. 8A and 8B, in which a clip 260 configured similarly as the clip 230 may have a curvature. The first jaw 262 and second jaw 264 may each have a first side surface 266, 268 that is concave with respect to the jaws 262, 264. A respective opposite side surface 270, 272 (marked in FIGS. 9B and 9C) may be convex with respect to the jaws 262, 264. Such a curvature may allow for contour to a shape of a left atrium. Features described in regard to the clip 30 may otherwise be utilized with the clip 260.

The features of the clip 260 may be utilized solely or in combination with any other example disclosed herein.

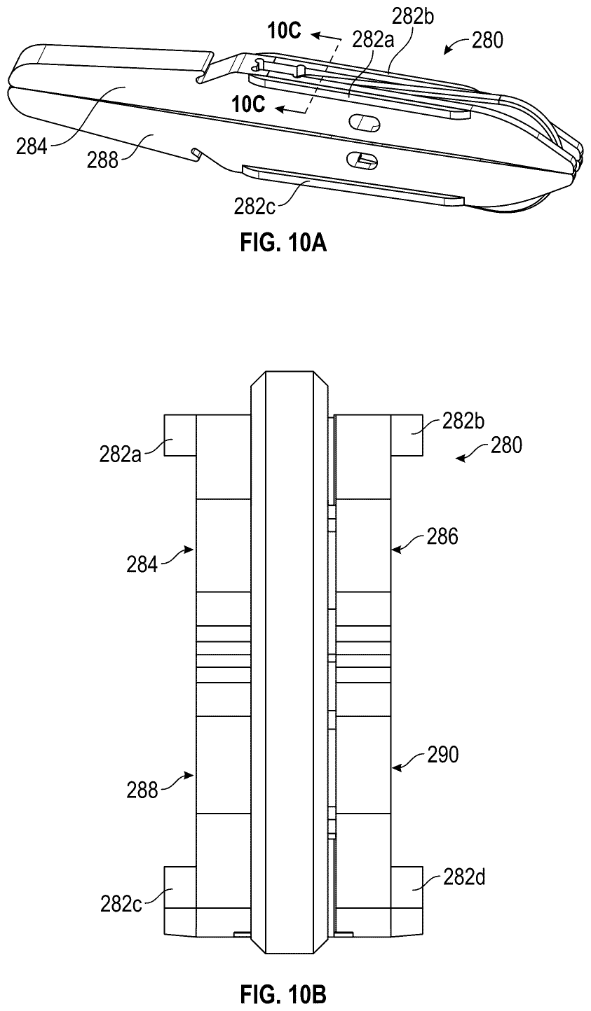

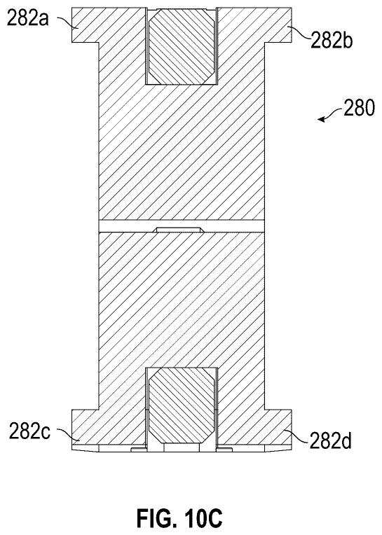

FIGS. 10A-10C illustrate an example of a clip 280 in which the elongate couplers 282a-d protrude from side surfaces 284, 286, 288, 290 that are flat. The side surfaces 284, 286, 288, 290 lack channels as shown in FIG. 2A for example. The expansion apparatus 122 may engage the couplers 282a-d protruding from the respective side surfaces 284, 286, 288, 290. FIG. 10B illustrates a proximal end view of the clip 280. FIG. 10C illustrates a side cross sectional view of the clip 280 along line 10C-10C in FIG. 10A. Features described in regard to the clip 30 may otherwise be utilized with the clip 280.

The features of the clip 280 may be utilized solely or in combination with any other example disclosed herein.

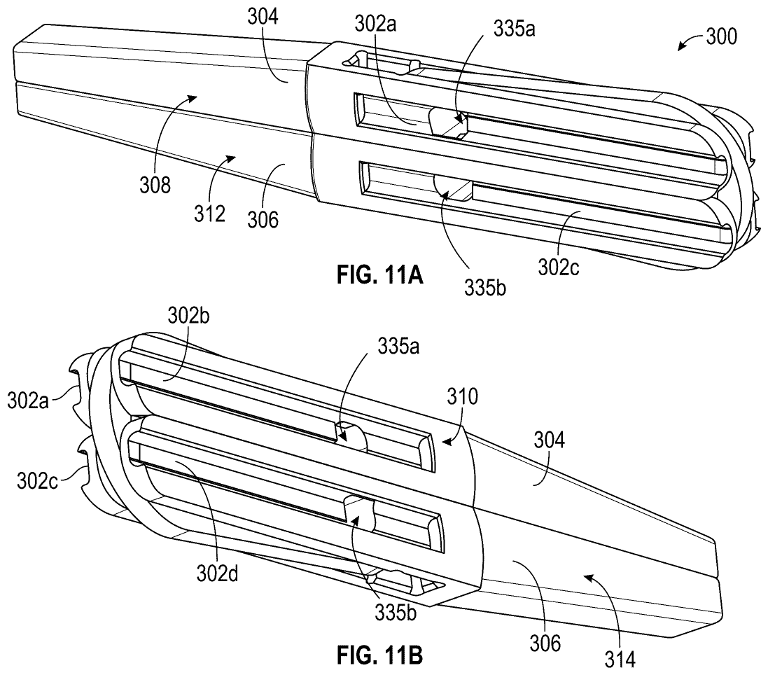

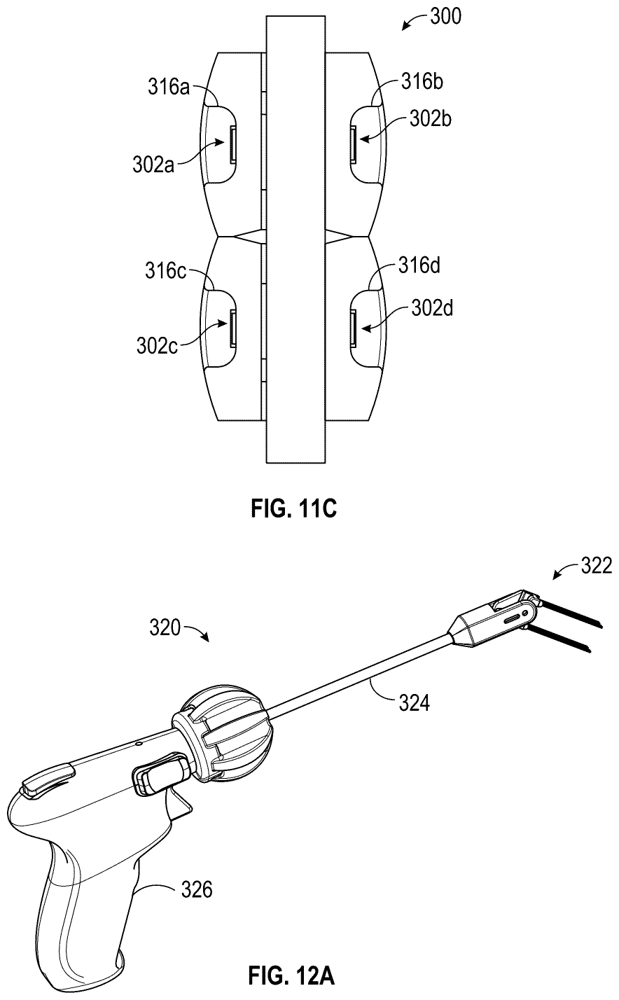

FIGS. 11A-11C illustrate an example of a clip 300 in which the elongate couplers 302a-d comprise channels extending along the length of the respective first jaw 304 and second jaw 306. The channels may comprise open channels exposed on the side surfaces 308, 310, 312, 314 of the respective first jaw 304 and the second jaw 306. Features described in regard to the clip 30 may otherwise be utilized with the clip 300.

FIG. 11C illustrates a proximal end view of the clip 300. Proximal end portions of the channels may comprise respective openings 316a-d of the first jaw 304 and second jaw 306 of the clip 300. The openings 316a-d may be on the end surfaces of the respective first jaw 304 and second jaw 306. The channels may be configured to receive arms of an expansion apparatus that may be utilized.

The features of the clip 300 may be utilized solely or in combination with any other example disclosed herein.

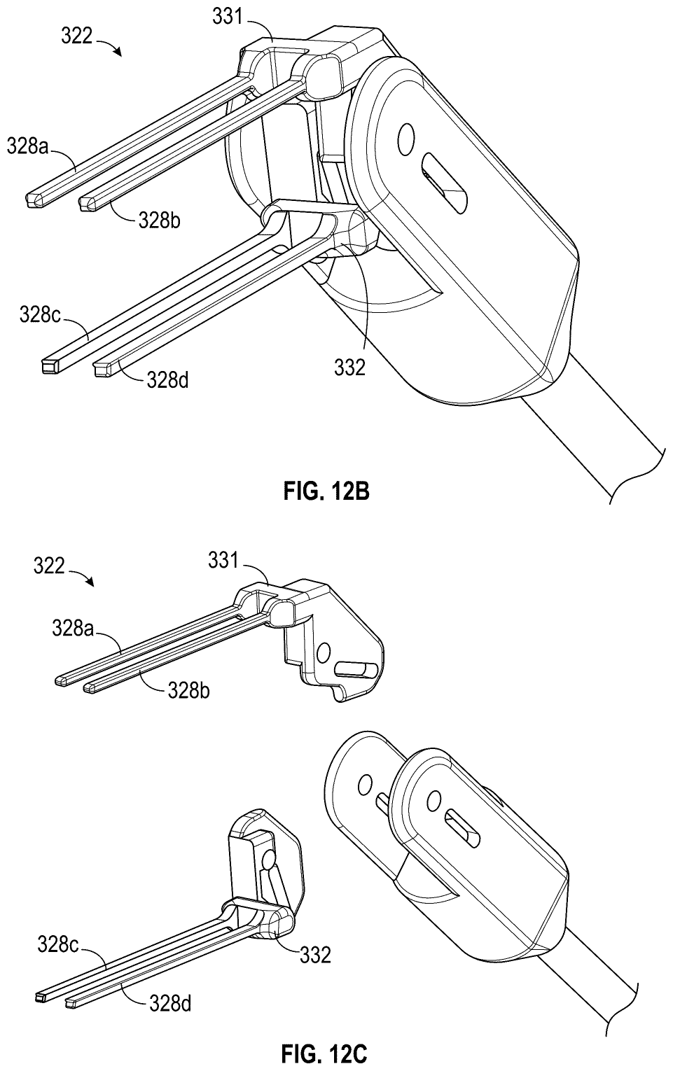

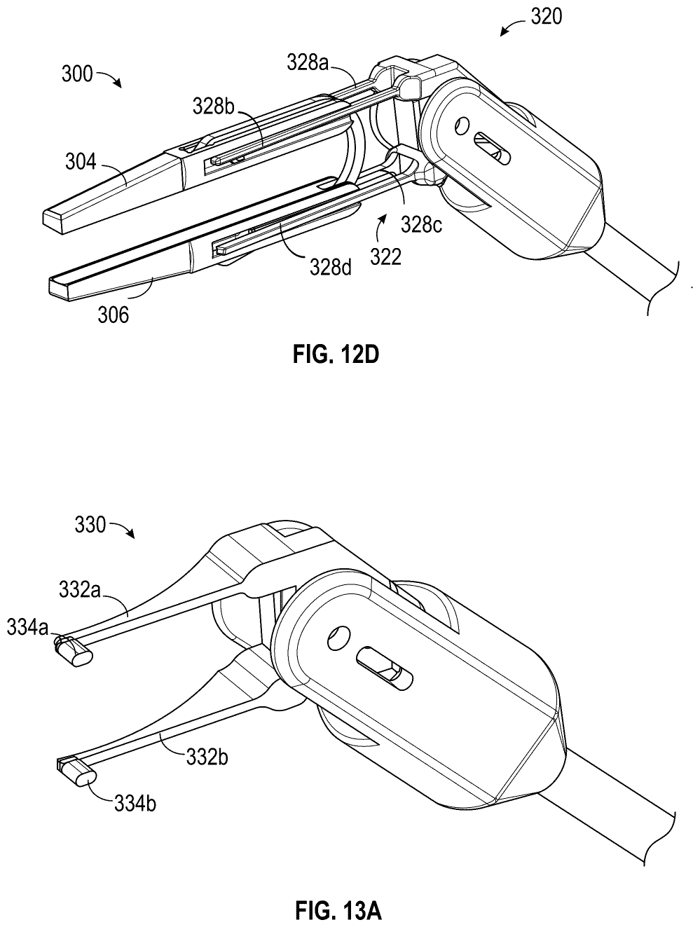

FIGS. 12A-12D illustrate an expansion apparatus 320 that is a variation of the expansion apparatus 122 described in regard to FIGS. 3A-3D. The expansion apparatus 320 may include features described in regard to the expansion apparatus 122 unless stated otherwise.

The expansion apparatus 320 may include an engagement portion 322 that may be positioned at the distal end portion of an elongate shaft 324. A handle 326 may be positioned at the proximal end portion of the elongate shaft 324. Components similar to the components of the expansion apparatus 122 may be utilized unless stated otherwise.

FIG. 12B illustrates a close up perspective view of the engagement portion 322. The engagement portion 322 may include four arms 328a-d that each may be configured to engage a respective elongate coupler 302a-d of a clip 300 as shown in FIG. 11A for example. The arms 328a-d may slidably engage with the elongate couplers 302a-d of the clip 300.

The upper arms 328a, b (e.g, a first arm 382a and a second arm 328b) may be configured to engage the elongate couplers 302a, b of the first jaw 304 and the lower arms 328c, d (e.g., a third arm 328c and a second arm 328d) may be configured to engage the elongate couplers 302c, d of the second jaw 306. The upper arms 328a, b may comprise forked ends of an upper base 331 of the engagement portion 322. The lower arms 328c, d may comprise forked ends of a lower base 332 of the engagement portion 322. The upper arms 328a, b may be configured to move relative to the lower arms 328c, d to open or close the clip 300. The upper arms 328a, b may be configured to perform movement simultaneous with each other, and the lower arms 328c, d may be configured to perform movement simultaneous with each other.

FIG. 12C illustrates an assembly view of the engagement portion 322. The arms 328a-d may be operated by an actuator assembly in the same manner as disclosed regarding the expansion apparatus 122 described in regard to FIGS. 3A-3D.

FIG. 12D illustrates a perspective view of the clip 300 engaged with the engagement portion 322 of the expansion apparatus 320. The arms 328a-d may slide into the elongate couplers 302a-d of the clip 300.

The features of the expansion apparatus 320 may be utilized solely or in combination with any other example disclosed herein.

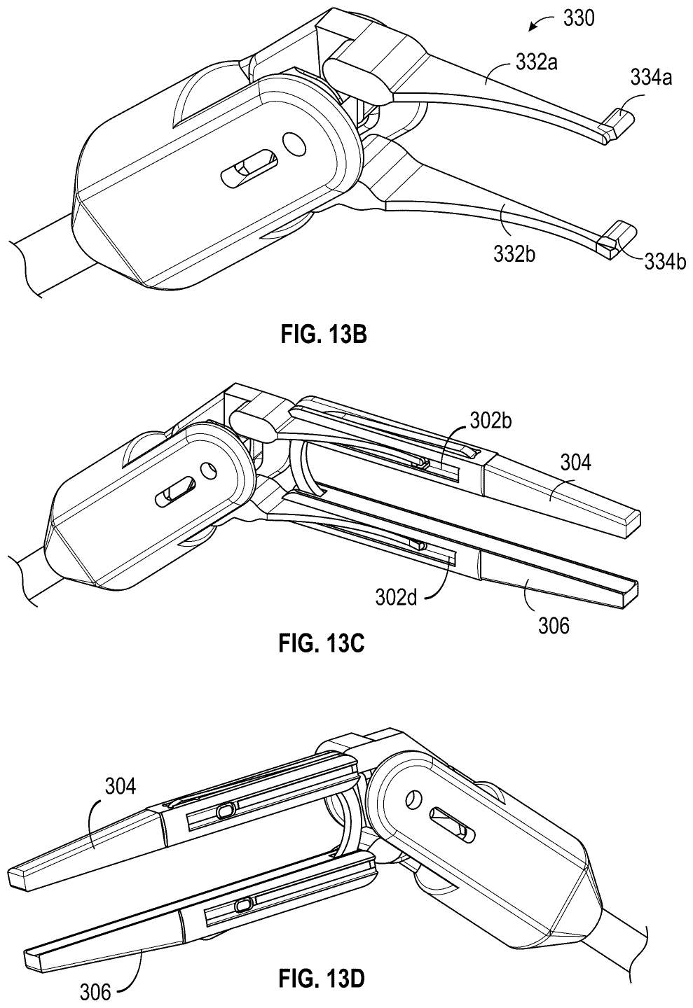

FIGS. 13A and 13B illustrate a variation of the engagement portion 322. The engagement portion 330 shown in FIG. 13A may include two arms 332a, b that may extend axially and respective protrusions 334a, b that may extend transverse to the length of the arms 332a, b. The protrusions 334a, b may be configured to engage recesses 335a, b (marked in FIG. 11B) in the respective first jaw 304 and second jaw 306 of the clip 300. FIGS. 13C and 13D, for example, illustrate such engagement. The arms 332a, b may extend along the length of the elongate couplers 302b, d and within the elongate couplers 302b, d.

The features of the engagement portion 322 may be utilized solely or in combination with any other example disclosed herein.

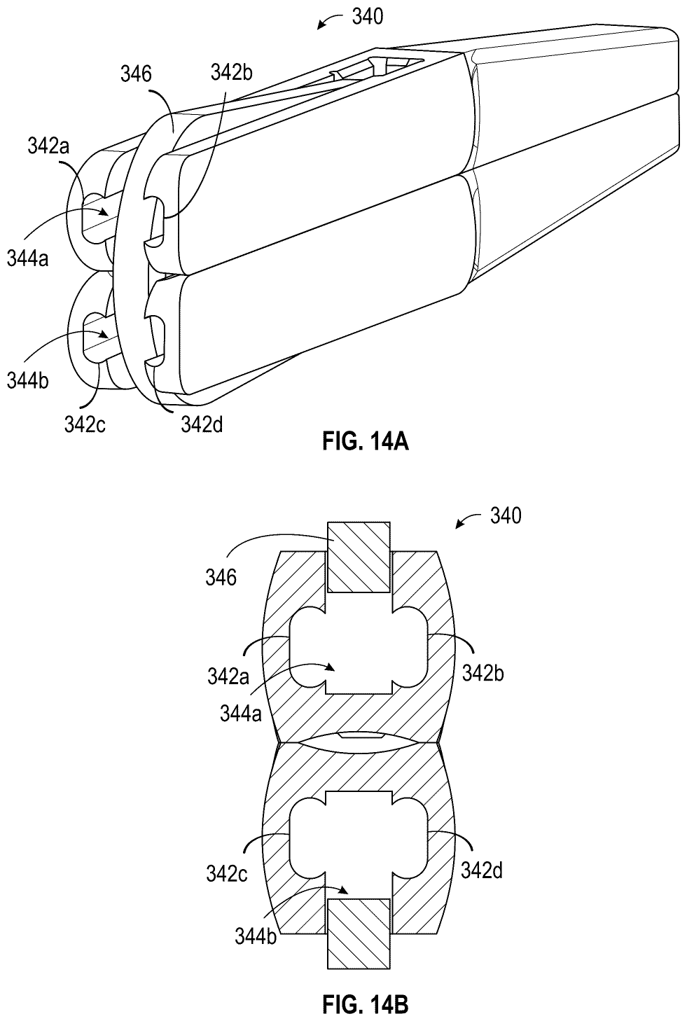

Variations in the configuration of the clip may be provided. FIGS. 14A and 14B, for example, illustrate a configuration of a clip 340 including elongate couplers 342a-d comprising opened channels that are opened to an interior channel 344a, b of the clip 340. FIG. 14B, for example, illustrates a side cross sectional view of the clip 340 along line 14B-14B in FIG. 14A. The elongate couplers 342a-d may be opened to the interior channels 344a, b that the spring 346 extends along. The clip 340 may otherwise include the features of the clip 300.

The features of the clip 340 may be utilized solely or in combination with any other example disclosed herein.

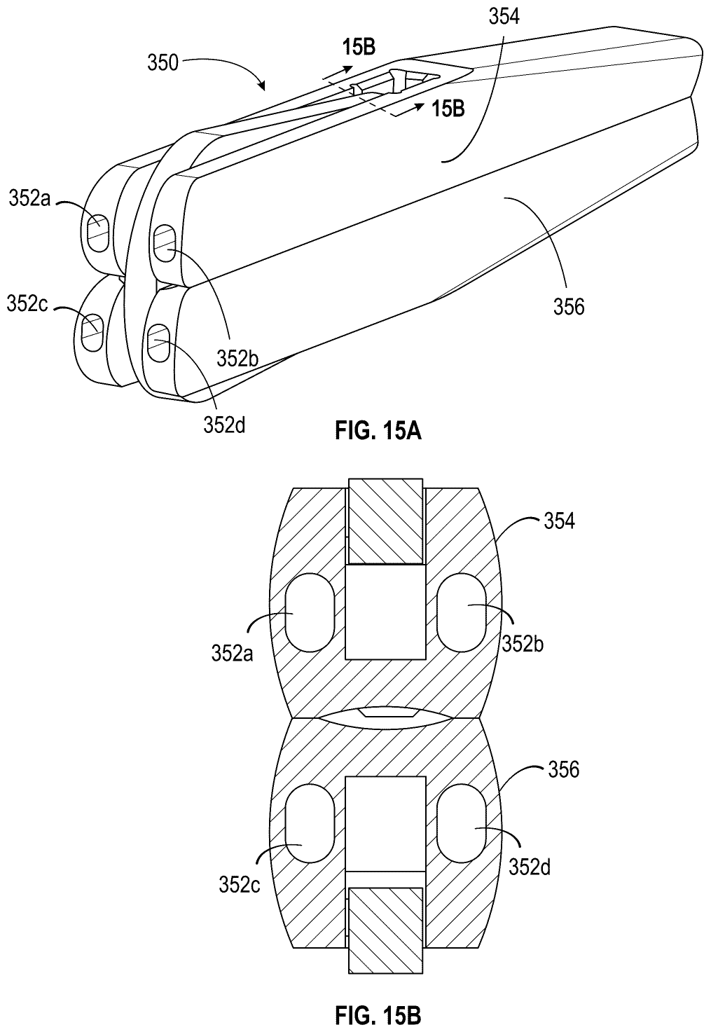

Variations in the configuration of the clip may be provided. FIGS. 15A and 15B, for example, illustrate a variation in which a clip 350 may include elongate couplers 352a-d that may comprise closed channels extending along a length of a respective one of the first jaw 354 and the second jaw 356. FIG. 15B, for example, illustrates a cross sectional view of the first jaw 354 and second jaw 356 along line 15B-15B in FIG. 15A. The channels of the elongate couplers 352a-d comprise closed channels within the jaws 354, 356. The clip 350 may otherwise include the features of the clip 300.

The features of the clip 350 may be utilized solely or in combination with any other example disclosed herein.

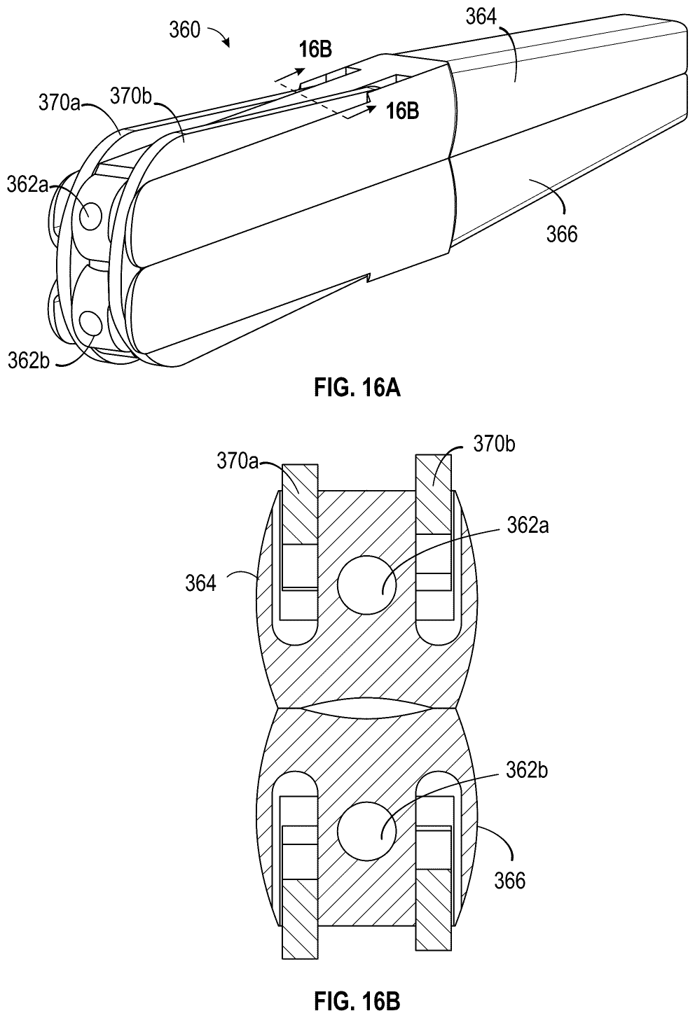

FIGS. 16A and 16B illustrate a configuration of a clip 360 including an elongate coupler 362a on the first jaw 364 and an elongate coupler 362b on the second jaw 366. The elongate couplers 362a, b may be disposed centrally within the respective first jaw 364 and the second jaw 366. FIG. 16B, for example, illustrates a cross sectional view of the first jaw 364 and the second jaw 366 along line 16B-16B in FIG. 16A. The channels of the elongate couplers 362a, b comprise closed channels within the jaws 364, 366.

In examples, the clip 360 may include two springs 370a, b that may be positioned on opposite sides of the respective elongate couplers 362a, b. The clip 360 may otherwise include the features of the clip 300.

The features of the clip 360 may be utilized solely or in combination with any other example disclosed herein.

Other configurations of clips may be utilized in examples.

Various other modifications of the clips disclosed herein may be provided.

Various other methods of deployment and use of the clips may be provided as desired.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The clips may be utilized solely or in methods disclosed herein, or in combination with other devices disclosed herein.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some examples have been described in connection with the accompanying drawings. The figures are drawn to scale, but such scale should not be limiting, since dimensions and proportions other than what are shown are contemplated and are within the scope of the disclosure. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, it will be recognized that any methods described herein may be practiced using any device suitable for performing the recited steps.

While a number of examples and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A clip for a portion of a heart, the clip comprising:
a first jaw extending from a first end portion to a second end portion along a length of the first jaw;
a second jaw extending from a first end portion to a second end portion along a length of the second jaw, the second jaw including a compression surface configured to face towards the first jaw, an outer surface facing opposite the compression surface, a first side surface extending from the compression surface to the outer surface, and a second side surface extending from the compression surface to the outer surface;
a spring configured to force the first jaw and the second jaw together to compress the portion of the heart with the first jaw and the second jaw; and
one or more elongate couplers positioned on one or more of the first side surface or the second side surface and extending along the length of the second jaw, the one or more elongate couplers configured to engage an expansion apparatus for the clip.

2. The clip of claim 1, wherein the one or more elongate couplers comprise one or more channels.

3. The clip of claim 2, wherein the one or more channels comprise closed channels or open channels.

4. The clip of claim 1, wherein the one or more elongate couplers comprise one or more protrusions.

5. The clip of claim 4, wherein the one or more protrusions form one or more rails extending along the length of the second jaw.

6. The clip of claim 4, wherein the one or more protrusions include an inner surface facing opposite the outer surface of the second jaw and towards a channel extending along the first side surface or the second side surface.

7. The clip of claim 1, wherein the second end portion of the first jaw and the second end portion of the second jaw form an open end of the clip, and the first end portion of the first jaw and the first end portion of the second jaw form a closed end of the clip, and the one or more elongate couplers are configured to engage the expansion apparatus advanced in a direction from the closed end of the clip towards the open end of the clip.

8. The clip of claim 1, wherein the one or more elongate couplers are configured to slidably engage with the expansion apparatus.

9. The clip of claim 1, wherein the spring includes a loop extending towards the first end portion of the first jaw and the first end portion of the second jaw.

10. The clip of claim 1, wherein the first jaw and the second jaw form a compression channel between the first jaw and the second jaw, the compression channel having an opening at the second end portion of the first jaw and the second end portion of the second jaw, and the compression channel being closed at the first end portion of the first jaw and the first end portion of the second jaw.

11. The clip of claim 10, wherein the spring closes the compression channel at the first end portion of the first jaw and the first end portion of the second jaw.

12. The clip of claim 1, wherein the first jaw includes:
a compression surface configured to face towards the second jaw,
an outer surface facing opposite the compression surface of the first jaw,
a first side surface extending from the compression surface of the first jaw to the outer surface of the first jaw, and
a second side surface extending from the compression surface of the first jaw to the outer surface of the first jaw and facing opposite the first side surface of the first jaw.

13. The clip of claim 12, further comprising one or more elongate couplers positioned on one or more of the first side surface of the first jaw or the second side surface of the first jaw and extending along the length of the first jaw, the one or more elongate couplers of the first jaw configured to engage an expansion apparatus for the clip.

14. The clip of claim 1, wherein the second jaw includes a first end surface positioned at the first end portion of the second jaw, and a second end surface positioned at the second end portion of the second jaw.

15. The clip of claim 14, wherein the one or more elongate couplers comprise an opening on the first end surface of the second jaw.

16. The clip of claim 14, wherein the second jaw includes a central portion and the one or more elongate couplers comprise one or more protrusions extending to a mid point between the central portion and the first end surface.

17. The clip of claim 1, wherein the second jaw includes a channel, the spring being positioned within the channel.

18. The clip of claim 17, wherein the channel includes a first side wall, a second side wall, a lower wall, and an upper portion that is open for the spring to pass through.

19. The clip of claim 1, wherein the first side surface is curved concave relative to the second jaw.

20. The clip of claim 1, wherein the clip is configured to occlude a left atrial appendage.
